(19) United States
(12) Patent Application Publication
(10) Pub. No.: US 2024/0277938 A1
(43) Pub. Date: Aug. 22, 2024

(54) INJECTION DEVICE

(71) Applicant: Future Injection Technologies Limited, Sheffield South Yorkshire (GB)

(72) Inventor: Kevin Stamp, Sheffield South Yorkshire (GB)

(21) Appl. No.: 18/287,976

(22) PCT Filed: Apr. 22, 2022
(86) PCT No.: PCT/EP2022/060710

§ 371 Date: Oct. 23, 2023

(30) Foreign Application Priority Data
Apr. 23, 2021 (GB) 2105845.8

(51) Int. Cl. A61M 5/20; A61M 5/32

(52) U.S. Cl. CPC A61M 5/2033; A61M 5/3245; A61M 5/326; A61M 2005/206

(57) ABSTRACT

This invention provides an improved automatic injection device comprising a syringe (having a needle), a barrel and a plunger, as well as driving means (e.g., a resilient member such as a coil spring) for driving the plunger into the barrel. The injection device is arranged to perform an automatic injection cycle in use comprising the stages of advancing the needle of the syringe for insertion, and driving the plunger into the barrel for dispensing a liquid contained in the barrel. The device is arranged such that during operation of the device, a driving force is transmitted from the driving means to the plunger during the dispensing stage. The device is arranged such that the syringe is driven forward during the needle advancement stage and the plunger is driven into the barrel during the dispensing stage for dispensing a liquid contained in the barrel.

### FIELD

The present invention relates to injection devices for the dispensing of liquids, and more particularly to automatic injection devices (so called “auto-injectors”).

### BACKGROUND

Automatic injection devices are typically arranged such that when activated by a user, e.g. by pressing a button etc., an injection is automatically delivered. In order to automatically deliver an injection, such devices may be arranged to automatically advance the needle of a syringe for insertion into the body of a user, and depress the plunger to dispense liquid from the syringe barrel. It is desired to improve various characteristics of such devices and how they operate.

Existing automatic injection devices include US2007265568A1 that includes tabs that prevent proximal motion of a needle shield after use and in a discard position. Other prior art includes WO2017/198383A1. Whilst these systems provide some mechanism for prevent motion of a needle shield, it is desired to improve the reliability of such mechanism and how it is integrated into the device as a whole.

### SUMMARY

The invention provides an improved automatic injection device comprising a syringe (having a needle), a barrel and a plunger, as well as driving means (e.g., a resilient member such as a coil spring) for driving the plunger into the barrel. The injection device is arranged to perform an automatic injection cycle in use comprising the stages of advancing the needle of the syringe for insertion, and driving the plunger into the barrel for dispensing a liquid contained in the barrel.

The device is arranged such that during operation of the device, a driving force is transmitted from the driving means to the plunger during the dispensing stage. The device is arranged such that the syringe is driven forward during the needle advancement stage and the plunger is driven into the barrel during the dispensing stage for dispensing a liquid contained in the barrel.

In a first aspect the device further comprises a needle sleeve configured to advance forward to an extended position in which it covers/surrounds the needle, as well as one or more locking members configured to lock the needle sleeve in its extended position at least subsequently to the dispensing stage to ensure that it remains in the extended position once the dose of liquid is dispensed from the barrel. This ensures that the needle sleeve remains in position to protect the needle, as well as a user/patient/caregiver. The needle sleeve is designed to protect the needle during and after operation, for example it may be configured to move only after activation of the automatic injection cycle (e.g., after the needle advancement stage has commenced).

The one or more locking members may also be separate to the needle sleeve (i.e., different pieces) and configured to translate from an initial position to a final position in which they lock the needle sleeve in its extended position. The use of separate, translating locking members is advantageous over rotating/bending tabs (e.g., US2007265568A1), since they allow more control over the mechanism. For example, they can be activated by axial movement of other parts of the device.

The needle sleeve may be configured to advance forward from a retracted position. The locking members may be further configured to lock the needle sleeve in its retracted position until commencement of the needle advancement stage. This adds a dual-function, providing further functionality not possible in the prior art (e.g., with tabs located on the sleeve itself). The locking members may, therefore, be configured in their initial position to lock the needle sleeve in its retracted position, and then to translate to their final position in which they lock the needle sleeve in its extended position.

The locking members may be configured to translate radially (e.g., radially inwards) from the initial position to the final position (and in contrast to the axially translating needle sleeve). In other words, the locking members may translate in a direction that is perpendicular to the direction of movement of the needle sleeve.

The locking members may be diametrically opposed, and/or spaced apart equally around the circumference of the device, for optimum balance. At least two locking members may be provided.

The automatic injection device may further comprise a syringe housing that is driven forwards upon action of the driving means during the needle advancement stage. The syringe housing may advantageously comprise one or more cam surfaces that are configured to contact and shift the locking members from their initial position (and, e.g., to their final position), as the syringe housing moves in an axial (forward) direction. This provides a convenient mechanism for shifting the locking members (e.g., so that they translate radially from the initial position to the final position as aforesaid).

The needle sleeve may comprise one or more flexing elements that are configured to move past the locking elements as the needle sleeve moves to its extended position, and then flex into alignment with the locking members once the needle sleeve is in its extended position, wherein such alignment locks the needle sleeve in its extended position as aforesaid.

The locking members may be additionally configured, in their final position, to limit forward axial movement of the syringe. They could therefore provide the further function of arresting the movement of the syringe, as well as controlling the movement of the needle sleeve.

In a second aspect of the invention, which may be claimed independently, the device comprises a cap that is configured to cover (e.g., at least partially) the dispending end thereof, as well as a rigid needle shield that fits over and protects the needle prior to operation. That is, the rigid needle shield is provided to protect the needle prior to operation (e.g., it is removed prior to the needle advancement stage), and is distinct from (for example) a needle sleeve as described above that is designed to protect the needle during and after operation.

In the second aspect the cap is configured to pull the rigid needle shield away from the device when it is itself removed from the device to expose the dispensing end ready for the automatic injection cycle to be performed. This provides a safe and reliable mechanism.

To achieve this in an advantageous manner, the cap may further comprise an annular end portion, as well as one or more arms that extend from the annular end portion into the device, the one or more arms each comprising teeth configured to latch onto and pull the rigid needle shield away from the device when the end cap is removed. This provides a highly reliable way of catching the rigid needle shield that ensures the rigid needle shield is pulled away from the device prior to operation.

The automatic injection device may further comprise a sleeve located around the arms of the cap, wherein the sleeve comprises one or more protrusions that are configured to urge the arms, and teeth thereof radially inwards as the end cap is removed to ensure that they latch onto the rigid needle shield and pull it away from the device as aforesaid. This further improves the reliability of the device to catch the rigid needle shield and ensure that it is removed when removing the end cap.

The end cap may comprise a cylindrical outer portion (e.g., surrounding the device and forming part of an initial, outer housing) that extends to the annular end portion, both of which cover a front portion of the device to cover the dispending end thereof prior to use.

In a third aspect, which may be claimed independently, the device further comprises a static member (i.e., it does not move (translate, etc.) by action of the driving means) that interacts with a rear end of the (movable) plunger to prevent relative movement between the static member and the plunger prior to operation of the automatic injection cycle. This ensures that the plunger is not depressed (and the injection administered or liquid lost) prior to starting the automatic injection cycle.

The device may comprise one or more lock members (distinct from the locking members described above with respect to the first aspect) configured to sit between the static member and plunger, so as to lock the plunger in place with the static member prior to activation of the automatic injection cycle. For example the lock members may be biased between the static member and the plunger, such that the plunger cannot move relative to the static member. Using distinct lock members in this manner provides a reliable way of ensuring that the plunger is not depressed prior to starting the automatic injection cycle.

The lock members may be configured to move at least partially in a radial direction out of alignment with the static member to release the plunger and allow activation of the automatic injection cycle. The static member may comprise a sleeve that sits concentrically outwards from the plunger.

Initially the lock members may be biased between the static member and the plunger to prevent relative axial movement therebetween.

The device may further comprise a movable abutment that sits radially inward of the lock members and is configured to move from a first, initial position in which it prevents inward radial movement of the lock members to a second position in which it permits inward radial movement of the lock members so that they move radially inwards and out of alignment with the static member, to release the plunger and allow activation of the automatic injection cycle. The moveable abutment can be configured to move axially between its first and second positions.

The lock members may be urged axially forwards by the plunger (e.g., due to the action of the driving means against the plunger) and against respective, radially extending cam surfaces of the static member. The cam surfaces may be configured, in turn, to urge the lock members radially inwards towards the movable abutment. This advantageously provides a tight fit for the locking components.

The driving means (e.g., resilient member or spring) may be configured to urge the plunger forwards, so as to press the plunger against the lock members and, in turn, press the lock members against the static member (e.g., cam surfaces thereof).

The lock members may be balls (e.g., ball bearings) configured to move radially within radially-extending, cylindrical cavities of the plunger.

The lock members may each comprise a magnetic material (or be magnetic, such as magnetic ball bearings) such that they are urged radially inwards and towards each other. Alternatively, or additionally the movable abutment may comprises a magnetic material such that the movable abutment attracts the lock members radially inwards. These magnetic embodiments are not essential for achieving the previously-described technical effects.

The first, second and third aspects are intended to be claimed separately (and independently), although the features thereof could be combined (for example, as shown below in the detailed description) in any combination to provide an improved automatic injection device.

Various embodiments of the invention are set out in the dependent claims.

### BRIEF DESCRIPTION OF THE DRAWINGS

Various embodiments will now be described, by way of example only, and with reference to the accompanying drawings in which:

FIG. 1 shows an automatic injection device 10 in accordance with the invention;

FIG. 2 shows a cross-section of the front end of the device of FIG. 1;

FIG. 3 shows another cross-section of the device of FIG. 1, which is oriented at 90 degrees from that shown in FIG. 2;

FIG. 4 shows a cross-section of the rear end of the device of FIG. 1; and

FIG. 5 shows a close up view of the rear end of the device of FIG. 1.

### DETAILED DESCRIPTION

Herewith will be described various embodiments of an automatic injection device as described above.

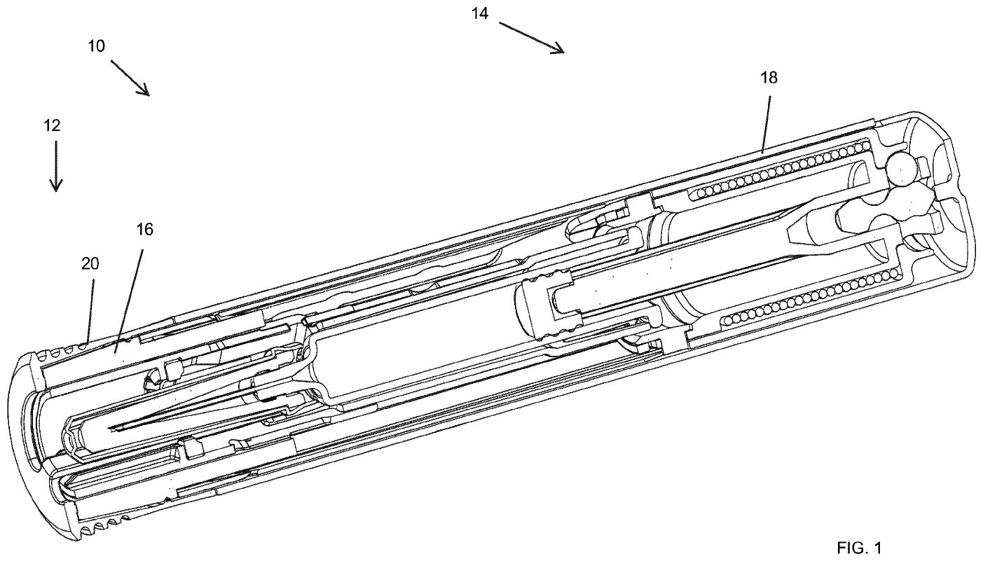

FIG. 1 shows an automatic injection device 10 in accordance with one embodiment of the invention, and prior to administering an injection using an automatic injection cycle.

The device 10 has a front, needle end 12 towards the left-hand side of the device in FIG. 1, and a rear end 14 towards the right-hand side. The device 10 comprises an end cap 20 at the front end 12, as well as front and rear outer housings 16, 18 that extend along the length of the device 10.

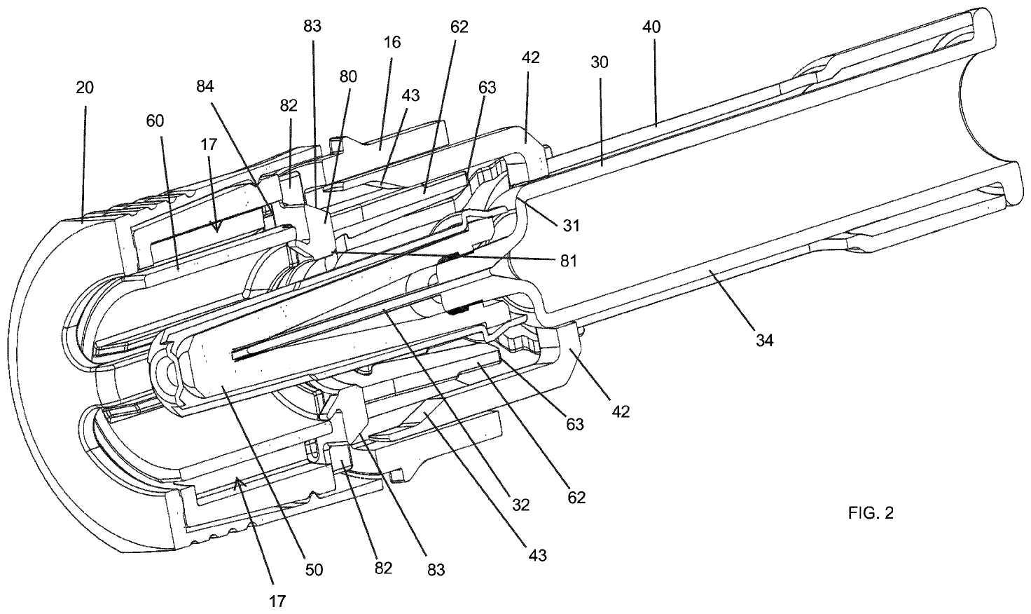

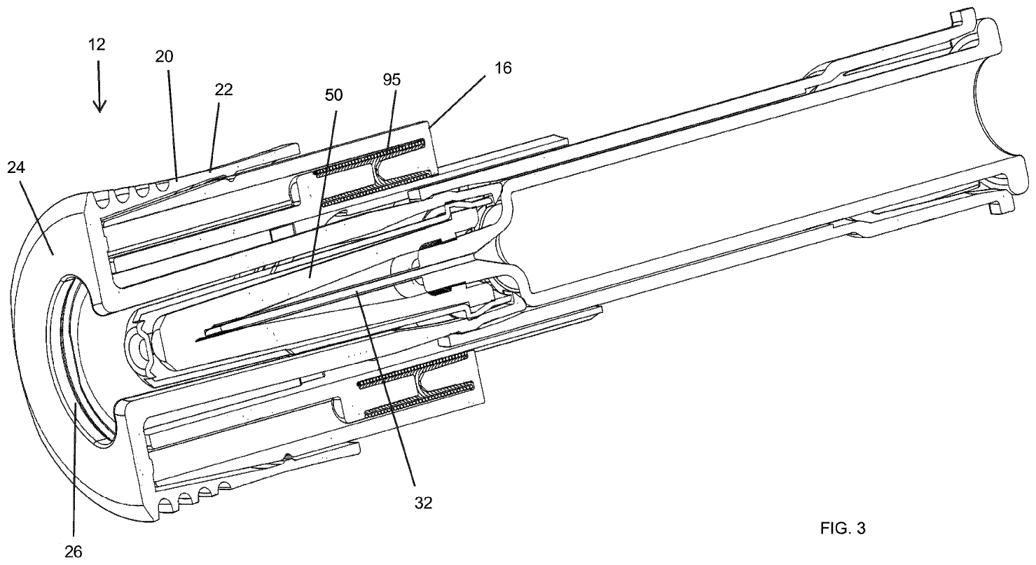

FIG. 2 shows a cross-sectional view of the front end 12, including a syringe 30 and a syringe housing 40 that extends around and covers the syringe 30. The syringe 30 comprises a needle 32 and a barrel 34, as well as a plunger 36 (FIG. 3) that is configured to move through the barrel 34 to administer an injection. The device 10 further comprises a rigid needle shield 50 (“RNS”) that is configured to protect and cover the needle 32 of the syringe 32.

The device 10 further comprises a needle sleeve 60 (which could also be referred to as a needle shield, but to avoid confusion with the rigid needle shield 50 is referred to generally herein as a sleeve), which is configured to protect the needle 32 and user/patient during and after the needle advancement and dispensing stages. The needle sleeve 60 extends around the rigid needle shield 50 initially, and sits within a portion of the syringe and front housings 40, 16.

A plurality of movable abutments or “collets” 80 are fitted within the device 10 and extend in a radial direction (relative to the longitudinal axis thereof) through each of the needle sleeve 60 and front housing 16. The collets 80 are configured to control advancement of the needle sleeve 60 and also prevent the cover 60 from retraction once the injection has been administered (to protect the needle 32).

Generally in a first aspect of the invention, the collets 80 are configured to translate in a radial direction due to their interaction with the syringe housing 40 and needle sleeve 60. The collets 80 are configured in a first radial position (as shown in FIG. 1) to prevent movement of the needle sleeve 60, and then move inwards to allow the needle sleeve 60 to advance with the needle 32 and protect/shield it. Once the injection has been administered, the collets 80 and needle sleeve 60 interact further so that the needle sleeve 60 remains in its extended position that covers and protects the needle 32. The operation of this will be described in more detail below

FIG. 3 shows a cross-section through the front end 12 of the device 10, which is oriented at 90 degrees from that shown in FIG. 2. The end cap 20 is shown and comprises a cylindrical outer portion 22 and a radially extending, annular end portion 24, both of which cover a portion of the front housing 16 and the end of the device 10. The end portion 24 extends radially from the cylindrical outer portion to a cylindrical inner rim 26. The end cap 20 comprises diametrically opposed arms 27 that extend from the cylindrical inner rim 26 back into the device 10.

According to a second aspect of the invention, the arms 28 of the end cap 20 are configured to pull the rigid needle shield 50 away from the needle 32 to expose it, when the end cap 20 is removed. This means that the needle 32 is ready to advance and administer an injection. Again, this will be described in more detail below.

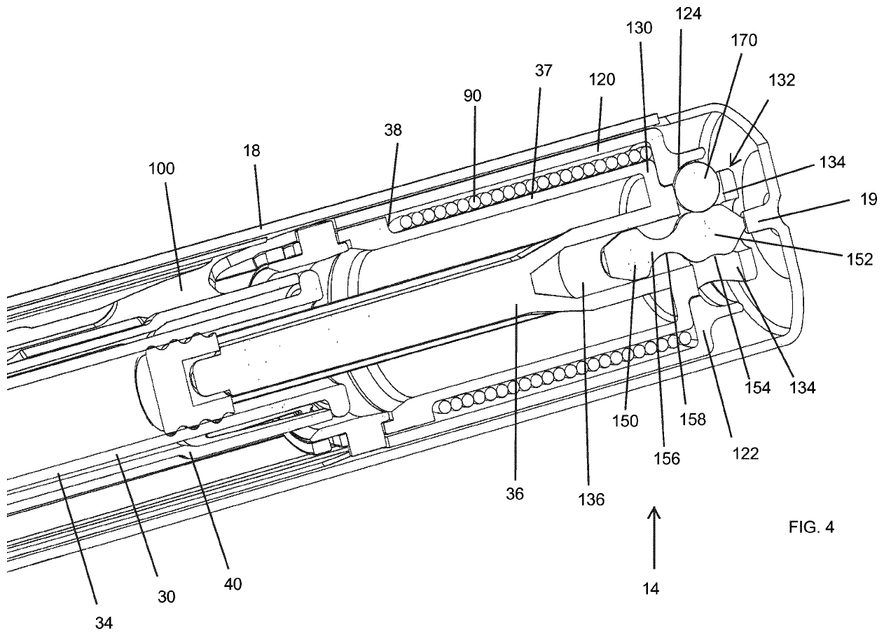

FIG. 4 shows a cross-section of the rear end 14 of the device 10, showing the rear housing 18 and parts of the device 10 within it.

The device 10 comprises a syringe guide 100 that is configured to control the advancement of the syringe 30 and its housing 40, as well as the transition between a needle advancement stage and dispensing stage of the automatic injection cycle. The barrel 34 is immobilised in the syringe housing 40 such that initially they are both moved within the device 10 by a force applied to the syringe housing 40.

The device 10 comprises a resilient member 90 (e.g., a coil spring, commonly referred to as the drive spring) that is configured to press against a rear extension 37 of the plunger 36, so as to urge the plunger 36 towards the front end 12 of the device 10. Because the rear extension 37 contacts the syringe guide 100 as well, it does not initially move through the barrel 34 to administer an injection.

An example of how to operate the syringe guide 100, syringe housing 40, and plunger 36 to administer an injection is described in PCT application number PCT/GB2010/000078, which is owned by the Applicant and incorporated herein by reference in its entirety. As this functionality is not critical to the present invention it will not be described in detail herein. Essentially, the drive spring is configured to act upon a drive cylinder arranged to selectively transmit a drive force to the plunger of the syringe. The movement is guided using various mechanisms (e.g., cam surfaces) to ensure that the needle is first advanced for insertion, after which the plunger is depressed for dispensing a dose of liquid from the barrel.

Although the previous application describes a step of retracting the needle, this is not required in the present invention since (as described herein) a needle sleeve/shield is advanced to cover the needle and protect it once a dose of liquid is dispensed from the barrel.

At the rear end 14 of the device 10 there is a mechanism for preventing the premature administering of an injection. The resilient member 90 is biased between a static member 120 and a shoulder 38 of the plunger rear extension 37.

In a third aspect of the invention the static member 120 interacts with a rear end 130 of the plunger 36 to prevent relative movement between the static member 120 and the plunger 36, prior to removal of the rear housing 18. It does this as illustrated using a shuttle 150 and ball 170 arrangement that will be described in more detail below.

Referring back to FIG. 2, as described above the first aspect of the invention is aimed at controlling the advancement of the needle sleeve 60 to extend and covers/protects the needle 32 (i.e., after administering of a dose of liquid from the barrel 34). The needle sleeve 60 may be urged forwards during the needle advancement stage of the automatic injection cycle, in which case it will be urged against the user's skin. The important feature in respect of the first aspect of the present invention, however, is that the needle sleeve 60 is initially prevented from axial movement, and then remains in its extended position to protect the needle once the dose of liquid is dispensed from the barrel. This dual-function is achieved by use of the collets 80 (“movable abutments”).

The collets 80 initially occupy a position that prevents the needle sleeve 60 from axial movement. To do this, the collets 80 comprise radially extending projections 82 that are configured to abut a radially extending surface of the front housing 16. Since the front housing 16 is generally static (i.e., does not move as part of the automatic injection cycle), it is prevented from axial movement, and so are the collets 80. In its initial, outer radial position, the needle sleeve 60 abuts the rearward-facing surface of the collets 80, preventing the needle sleeve 60 from axial movement as well.

Upon activation of the automatic injection cycle, the syringe 30 and its housing 40 will move axially towards the front end 12 of the device 10 in the needle advancement stage.

The syringe housing 40 comprises axially extending arms 42 that are each circumferentially aligned with a respective collet 80. The arms 42 each comprise a cam surface 43 that faces an opposing surface 83 on a respective collet 80. The surfaces 43, 83 are angled so that as the arms 42 move towards and contact the collet 80, the surfaces 83 on each collet 80 ride up the opposing surfaces 43 on each arm 42, causing the collet 80 to move radially inwards.

As the collets 80 translate radially inward, the rearward-facing surface of the collets 80 moves out of alignment with the needle sleeve 60, meaning that the needle sleeve 60 can now move axially forward (with the collets 80 remaining in a fixed axial position).

To cause axial movement of the needle sleeve 60, a resilient member 95 (see FIG. 3) may be biased between the needle sleeve 60 and the front housing 16. This causes the needle sleeve 60 to advance forwards once it is able to do so, i.e., once the collets 80 translate from their initial, outer radial position to their inner radial position. In advancing forwards, the needle sleeve 60 covers and protects the needle 32 of the syringe 30. This is both to avoid damaging the needle 32, but also to protect a user/patient once the needle 32 is removed after dispensing.

The collets 80 and needle sleeve 60 interact again to ensure that the needle sleeve 60 remains advanced once the injection is administered. That is, the needle sleeve 60 is configured to move axially forwards whilst the collets 80 remain in the same axial position. Once the needle sleeve 60 has moved forwards a predetermined amount, one or more portions of the needle sleeve 60 are configured to catch onto the collet 80 again. In doing so, the needle sleeve 60 is once more locked against axial movement by the collets 80.

In the illustrated embodiment this is achieved using resilient arms 62 that are located on the needle sleeve 60, which are each configured to ride along a respective radially extending (and circumferentially facing) surface 84 on the sides of each collet 80. As they do so, the resilient arms 62 are configured to flex/bend away from the surface 84 to allow the axial movement. The resilient arms 62 could more generally be referred to as flexing elements.

Once the needle sleeve 60 has moved axially by the predetermined amount, the arms 62 move past the collets 80 and flex back to their original positions. Upon doing so, the ends 63 of the arms 62 move into circumferential alignment with the collets 80. This prevents the arms 62 (and needle sleeve 60) from moving rearwards, and ensures that the needle sleeve 60 remains in its advanced, protective position.

The syringe 30 advances forward with the syringe housing 40, and the collets 80 may have the additional function of limiting forward axial movement of the syringe 30 once they are in their final position. That is, the collets 80 move radially inwards from their position shown in FIG. 2 as described above, and in their inner radial position block the path of the syringe 30. More specifically, a radially inner surface 81 of the collets 80 is configured to abut a shoulder 31 of the syringe 30 once the syringe 30 advances a sufficient distance (i.e., a distance that allows an injection to be administered via the needle 32). This prevents axial movement of the syringe 30 as a result.

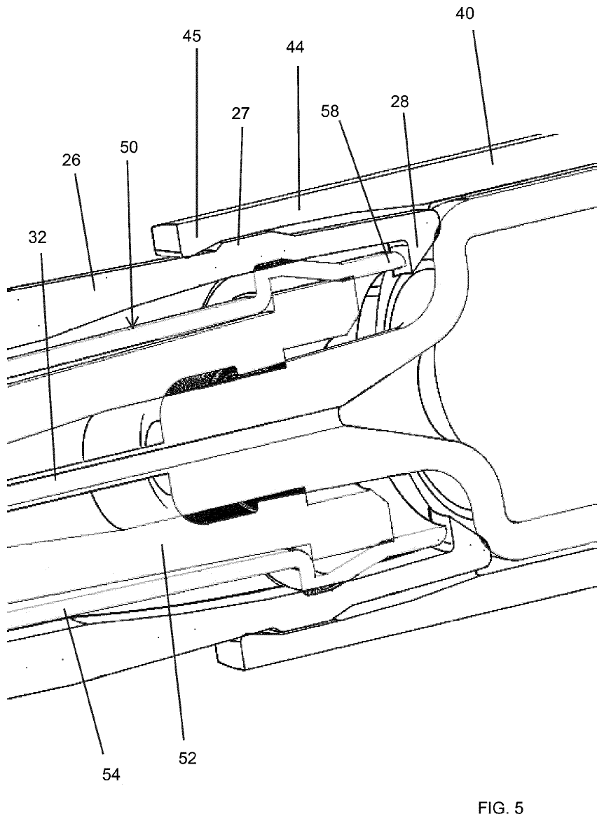

Discussing now the second aspect of the invention, FIG. 5 shows a close up view of the interaction between the end cap 20 and the rigid needle shield 50. This interaction ensures that the rigid needle shield 50 pulls away from the needle 32 to expose it when the end cap 20 is removed. The illustrated rigid needle shield 50 comprises an inner sheath 52 and an outer sheath 54, although this is not essential and it could, for example be provided as a single piece (or more pieces).

It is important to ensure that the rigid needle shield 50 is completely removed from the device 10 when the end cap 20 is removed. This exposes the needle 32 ready for advancing in the automatic injection cycle.

The axially extending arms 27 of the end cap 20 each comprise a tooth 28 that is configured to overhang an end 58 of the rigid needle shield 50, and as illustrated an annular end 58 of the outer sheath 54. The teeth 28 are configured to latch onto the end 58 and pull the rigid needle shield 50 away from the device 10 when the end cap 20 is removed.

To optimise this arrangement, the syringe housing 40 and the end cap 20 interact to urge the teeth 28 towards the rigid needle shield 50 as the end cap 20 is removed from the device 10. The syringe housing 40 comprises second axially extending arms 44 (which are distinct from the arms 42 described above with reference to the first aspect).

Each of the second arms 44 comprises an end 45 having an increased thickness portion as compared to the remainder of the arm 44. The end cap 20 comprises the one or more axially extending arms 27 that extend from the cylindrical inner rim 26 of the end cap 20 backwards into the device 10. The arms 27 initially reside within the reduced thickness portion of the arm 44 of the syringe housing 40.

When the end cap 20 is removed from the device 10, the arm(s) 27 move axially past the increased thickness portions at the end 45 of the second arms 44. As a result, the cylindrical inner portion 26, including the teeth 200 are urged radially inwards to ensure that they latch onto the rigid needle shield 50 and pull it away from the device 10.

Moving now to the third aspect of the invention and FIG. 4, this illustrates the rear end 14 of the device 10. As noted above, in this aspect a mechanism is provided to prevent an injection being administered prematurely. More specifically, the static member 120 interacts with a rear end 130 of the plunger 36 to prevent relative movement between the static member 120 and the plunger 36, prior to removal of the rear housing 18.

The rear end 130 of the plunger 36 comprises an axially extending section 132 that comprises a plurality of circumferential cavities 134, which are oriented so that each has a central axis perpendicular to the longitudinal axis of the device 10. Within each cavity 134 sits a ball 170, although one of the cavities is shown without a ball in FIG. 4 for illustrative purposes. Due to the orientation of the cavities 134, the balls 170 may move in a radial direction. However, this is strictly controlled using the other portions of the device 10, as follows.

The rear housing 18 is attached to a shuttle 150, which is configured to move axially within a cylindrical cavity 136 oriented parallel to the longitudinal axis of the device 10 (and perpendicular to the cavities 134 of the plunger rear end 130). The shuttle 150 is connected initially to a portion 19 of the rear housing 18, such that removal of the rear housing 18 from the device 10 moves the shuttle in an axial direction. The connection is not permanent (e.g., friction, interference fit) so that the shuttle 150 can detach from the rear housing 18 upon a force pulling it in the opposite direction.

The shuttle 150 comprises a first portion 152 having a slight annular indentation 154, which initially receives the balls 170 as shown in FIG. 4. In this position, the resilient member 90 urges the plunger 36 forwards, and since the balls 170 sit within the cavities 134, they will also be urged in the same direction. To prevent movement of the plunger 36 in this direction, the static member 120 comprises a radially extending annular portion 122 that extends past the balls 170. A sloping, rearward-facing surface 124 of this portion 122 is configured to abut the balls 170 and prevent them from moving axially past the static member 120.

In order to permit movement of the plunger 36 in the axial direction, it will be appreciated that the balls 170 have to move radially inwards. As illustrated, this is achieved by providing the shuttle 150 with a second portion 156 having an increased annular indentation 158. This is sized such that the balls 170 occupying the increased indentation 158 will be able to move radially inwards far enough so that they are out of alignment with the rearward-facing surface 124, meaning that this no longer prevents them from moving axially.

To move the balls 170 into alignment with the second portion 156 of the shuttle 150, the rear housing 18 can be removed from the device 10. This pulls the shuttle 150 rearwards, such that the balls 170 align with the second portion 156 and move radially inwards. This movement is ensured by the rearward-facing surface 124 of the static member 120 having its sloping (or a curved/angled, etc.) geometry.

That is, the force of the resilient member 90 causes the balls 170 to be pressed against the rearward-facing surface 124 by the rear surface of the cavities 134 within which they are held. Accordingly, as soon as they are permitted to do so (by the axial movement of the shuttle 150) they will ride up the slope of the rearward-facing surface 124 and move radially inwards into the increased indentation 158 of the shuttle 150. Upon doing so, the shuttle 150 will be prevented from further axial movement, since the balls 170 lock it in position relative to the plunger 36 (since they remain within the cavities 134), and it will detach from the rear housing 18.

Once the balls 170 reside within the increased indentations 158, they will move out of alignment with the static member 120 and allow axial movement of the plunger 36.

To assist this operation further, the balls 170 and/or the shuttle 150 may be made of a magnetic material, and configured to attract radially inwards towards the longitudinal axis of the device 10. For example, the cavities 134 and balls 170 could be arranged symmetrically on either side of the longitudinal axis, meaning that they will attract towards each other in use. This also means the balls 170 will attract to each other over the shuttle 150 and facilitate ease of assembly.

It will be appreciated that initially (e.g., during the needle advancement stage of the device 10) the plunger 36 moves together with the syringe guide 100, syringe 30 and housing 40, and cannot move relative to the syringe 30 to expel its contents.

The three aspects of the present invention may be claimed separately, or combined (in any permutation) to provide an improved automatic injection device.

Although the present invention has been described with reference to various embodiments, it will be understood by those skilled in the art that various changes in form and detail may be made without departing from the scope of the invention as set forth in the accompanying claims.

1. An automatic injection device comprising:
a syringe having a needle;
a barrel;
a plunger; and
driving means (e.g., a resilient member or spring) for driving the plunger into the barrel,
wherein the injection device is arranged to perform an automatic injection cycle in use comprising the stages of advancing the needle of the syringe for insertion, and driving the plunger into the barrel for dispensing a liquid contained in the barrel,
wherein the device is arranged such that during operation of the device, a driving force is transmitted from the driving means to the plunger during the dispensing stage,
wherein the device is arranged such that the syringe is driven forward during the needle advancement stage and the plunger is driven into the barrel during the dispensing stage for dispensing a liquid contained in the barrel,
wherein the device further comprises a needle sleeve configured to advance forward to an extended position in which it covers/surrounds the needle, as well as one or more locking members configured to lock the needle sleeve in its extended position at least subsequently to the dispensing stage to ensure that it remains in the extended position once the dose of liquid is dispensed from the barrel,
wherein the one or more locking members are separate to the needle sleeve and configured to translate from an initial position to a final position in which they lock the needle sleeve in its extended position.

2. An automatic injection device as claimed in claim 1, wherein the needle sleeve is configured to advance forward from a retracted position, wherein the locking members are further configured to lock the needle sleeve in its retracted position until commencement of the needle advancement stage.

3. An automatic injection device as claimed in claim 2, wherein the locking members are configured in their initial position to lock the needle sleeve in its retracted position, and then to translate to their final position in which they lock the needle sleeve in its extended position.

4. An automatic injection device as claimed in claim 3, wherein the locking members are configured to translate radially inwards from the initial position to the final position.

5. An automatic injection device as claimed in any preceding claim, further comprising a syringe housing that is driven forwards upon action of the driving means during the needle advancement stage, wherein the syringe housing comprises one or more cam surfaces that are configured to contact and shift the locking members from their initial position (and, e.g., to their final position).

6. An automatic injection device as claimed in any preceding claim, wherein the needle sleeve comprises one or more flexing elements that are configured to move past the locking elements as the needle sleeve moves to its extended position, and then flex into alignment with the locking members once the needle sleeve is in its extended position, wherein such alignment locks the needle sleeve in its extended position as aforesaid.

7. An automatic injection device as claimed in any preceding claim, wherein the locking members are additionally configured, in their final position, to limit forward axial movement of the syringe.

8. An automatic injection device comprising:
a syringe having a needle;
a barrel;
a plunger; and
driving means (e.g., a resilient member or spring) for driving the plunger into the barrel,
wherein the injection device is arranged to perform an automatic injection cycle in use comprising the stages of advancing the needle of the syringe for insertion, and driving the plunger into the barrel for dispensing a liquid contained in the barrel,
wherein the device is arranged such that during operation of the device, a driving force is transmitted from the driving means to the plunger during the dispensing stage,
wherein the device is arranged such that the syringe is driven forward during the needle advancement stage and the plunger is driven into the barrel during the dispensing stage for dispensing a liquid contained in the barrel,
wherein the device comprises a cap that is configured to cover the dispending end thereof, as well as a rigid needle shield that fits over and protects the needle prior to operation,
wherein the cap is configured to pull the rigid needle shield away from the device when it is itself removed from the device to expose the dispensing end ready for the automatic injection cycle to be performed,
wherein the cap comprises an annular end portion, as well as one or more arms that extend from the annular end portion into the device, the one or more arms each comprising teeth configured to latch onto and pull the rigid needle shield away from the device when the end cap is removed.

9. An automatic injection device as claimed in claim 8, further comprising a sleeve located around the arms of the cap, wherein the sleeve comprises one or more protrusions that are configured to urge the arms, and teeth thereof radially inwards as the end cap is removed to ensure that they latch onto the rigid needle shield and pull it away from the device as aforesaid.

10. An automatic injection device as claimed in claim 8 or 9, wherein the end cap comprises a cylindrical outer portion that extends to the annular end portion, both of which cover a front portion of the device to cover the dispending end thereof.

11. An automatic injection device comprising:
a syringe having a needle;
a barrel;
a plunger; and
driving means (e.g., a resilient member or spring) for driving the plunger into the barrel,
wherein the injection device is arranged to perform an automatic injection cycle in use using a plurality of axially movable components, wherein the automatic injection cycle comprises the stages of advancing the needle of the syringe for insertion, and driving the plunger into the barrel for dispensing a liquid contained in the barrel,
wherein the device is arranged such that during operation of the device, a driving force is transmitted from the driving means to the plunger during the dispensing stage,
wherein the device is arranged such that the syringe is driven forward during the needle advancement stage and the plunger is driven into the barrel during the dispensing stage for dispensing a liquid contained in the barrel,
wherein the device further comprises a static member that interacts with a rear end of the plunger to prevent relative movement between the static member and the plunger prior to operation of the automatic injection cycle.

12. An automatic injection device as claimed in claim 11, wherein the device comprises one or more lock members configured to sit between the static member and plunger, so as to lock the plunger in place with the static member prior to activation of the automatic injection cycle.

13. An automatic injection device as claimed in claim 12, wherein the lock members are configured to move radially out of alignment with the static member to release the plunger and allow activation of the automatic injection cycle.

14. An automatic injection device as claimed in claim 12 or 13, wherein the static member comprises a sleeve that sits concentrically outwards from the plunger,
wherein initially the lock members are biased between the static member and the plunger to prevent relative axial movement therebetween,
wherein a movable abutment sits radially inward of the lock members and is configured to move from a first, initial position in which it prevents inward radial movement of the lock members to a second position in which it permits inward radial movement of the lock members so that they move radially inwards and out of alignment with the static member, to release the plunger and allow activation of the automatic injection cycle.

15. An automatic injection device as claimed in claim 14, wherein the lock members are urged axially forwards by the plunger and against a radially extending cam surface of the static member, which cam surface is configured, in turn, to urge the lock members radially inwards towards the movable abutment.

16. An automatic injection device as claimed in claim 15, wherein the driving means is configured to urge the plunger forwards.

17. An automatic injection device as claimed in claim 14, 15 or 16, wherein the lock members are balls configured to move radially within radially-extending, cylindrical cavities of the plunger.

18. An automatic injection device as claimed in any of claims 12-17, wherein the lock members each comprise a magnetic material such that they are urged radially inwards and towards each other.

19. An automatic injection device as claimed in any of claims 14-17, wherein the lock members comprise a magnetic material and the movable abutment comprises a magnetic material such that the movable abutment attracts the lock members radially inwards.
